(19) United States
(12) Patent Application Publication
(10) Pub. No.: US 2010/0030033 A1
(43) Pub. Date: Feb. 4, 2010

(54) SURGICAL LIGHT APPARATUS

(75) Inventors: Daniel K. Farley, Traverse City, MI (US); Steve Nowak, Traverse City, MI (US)

Correspondence Address: MCANDREWS HELD & MALLOY, LTD, 500 WEST MADISON STREET, SUITE 3400, CHICAGO, IL, 60661, US

(73) Assignee: THOMPSON SURGICAL INSTRUMENTS, Traverse City, MI (US)

(21) Appl. No.: 12/185,620

(22) Filed: Aug. 4, 2008

(51) Int. Cl. A61B 1/07

(52) U.S. Cl. 600/249

(57) ABSTRACT

A surgical light apparatus for illuminating a surgical site. The light apparatus may include a light housing, fiber optical cable, a sheath, and a cable connector. The fiber optical cable may be comprised of a bundle of fiber optical cables that have distal and proximate ends. The proximate end may be operably connected to the cable connector, and oriented to receive light emitted from a light source. The distal end of the fiber optical cable may be operably connected to the light housing. The light housing may include a main body and a cover, which either individually, or in combination with each other, provides an outlet at which the distal ends of the fiber optical cables may be dispersed. The light housing may include, or be operably connected to, an attachment mechanism that may attach the surgical light apparatus to a surgical instrument, for example a retractor blade.

### BACKGROUND OF THE INVENTION

Embodiments of the present invention generally relate to a light source for use in illuminating a surgical site. More specifically, embodiments of the present invention relate to a reusable surgical light apparatus that may be removably attached to a surgical instrument and be positioned in close proximity to a surgical site.

Proper illumination of a surgical site is often a critical aspect of surgery. The lighting of the surgical site often entails the placement of light sources that not only sufficiently illuminate the surgical site, but also minimize the potential for the surgical site to be obscured by shadows created by the placement and/or movement of the surgeon and other members of the surgical team during surgery. Accordingly, the ability to place a light source in close proximity to the surgical site may reduce the risk that these undesirable shadows may be created during surgery. But the lights used during surgery typically generate, or operate at, relatively high temperatures. Moreover, if placed too close to the surgical site, the heat generated by such lights may burn the patient or cause other undesirable cell damage.

Accordingly, lights, or light emitting equipment, are generally placed at a distance away from the patient so as to prevent the patient from either being burned or experiencing other forms of cell damage. For example, a light or light emitting device may be mounted to the surgeon's head or to a headpiece worn by the surgeon. Further, lights may be positioned above the surgeon or around the surgeon. Yet, such lights or light emitting devices typically must be sized to compensate for the distance between these apparatuses and the surgical site. For example, a surgeon's headlight may typically only come within approximately two feet of the surgical site. Therefore, the size and/or the power used to operate and/or emit the light source of the headlight must be configured to accommodate both the distance the light needs to travel from the light source to the surgical site, and the brightness of the illumination needed to be delivered to the surgical site. But by increasing the size of the light source and/or the power used to operate the light source, the amount of heat generated by the light source and transmitted from the headlight may also increase. Moreover, the heat generated by and/or transmitted from the light source may add to the discomfort a surgeon may experience when wearing the headlight. Additionally, other surgeons or staff that are assisting during the surgery that are not wearing a headlight may not benefit from the surgeon's headlight during periods in which the surgeon wearing the headlight is not looking at the surgical site. Accordingly, such situations may increase the chances that the surgical site may be at least partially obscured by shadows.

### BRIEF SUMMARY OF THE INVENTION

Embodiments of the present invention relate to a surgical light apparatus that may be removably attached to a surgical instrument. More specifically, embodiments of the present invention generally relate to a bundle of reusable and light transmitting fiber optical cables that may be attached to a surgical instrument and be placed in close proximity to a surgical site.

According to certain embodiments, the surgical light apparatus of the present invention may include a light housing, a bundle of fiber optical cables, and a cable connector. The bundle of fiber optical cable includes a proximate end and a distal end. The proximate end of the bundle of fiber optical cables may be operably connected to the cable connector. Further, the proximate end of the bundle of fiber optical cables may be generally oriented in, or by, the cable connector so as to receive light that is emitted from a light source. For example, the cable connector may be configured to operably connect or couple the proximate end of the bundle of fiber optical cables to a lamp housing that houses the light source, or to another cable that is transmitting light emitted from the light source. According to certain embodiments, the light source may be a light bulb, for example a xenon light bulb.

The distal end of the bundle of fiber optical cables may be housed in the light housing. The light housing may include an outlet, about which the distal end of the fiber optical cables may be positioned and/or dispersed. For example, according to certain embodiments, the light housing may include an outlet, such as a slot, pocket, or opening, among others, that orients and/or positions the distal end of the fiber optical cable about a core area of the light housing. According to some embodiments, the light housing may include a main body and a cover. The main body and/or cover may be configured so that the main body or housing either separately, or when joined together, provide an outlet that may have a slot configuration through which the exposed distal ends of the fiber optical cables may be placed so as to emit the transmitted light to the surgical site.

The outlet may have a variety of geometric configurations, including, but not limited to, generally circular, rectangular, square, linear, non-linear, triangular, or oval. According to certain embodiments, the outlet may have a generally circular, oval, or elliptical shape that surrounds an inner core area, such as, for example, creating a ring around the inner area. Further, the outlet may be comprised of one or more than one opening in the light housing. Additionally, the outlet may be sized so that only a portion of the outlet is occupied by the light emitting portion of the distal ends of the fiber optical cables. Moreover, the outlet may be configured to allow at least a portion of the bundle of fiber optical cables to be dispersed along the outlet. This dispersal of the fiber optical cables may assist in dispersing the heat generated or transmitted at the distal end of the fiber optical cables while light is being emitted from the fiber optical cables. According to some embodiments, filler material may also be included in the outlet, and may also be used in dispersing the bundle of fiber optical cables about the outlet so that the distal ends of the fiber optical cables may operate at lower temperatures when light is being emitted from the fiber optical cables.

According to certain embodiments, the fiber optical cables may include at least one gap between the proximate end and the distal end of the cables. This gap may create an air space between adjacent first and second surfaces of the fiber optical cables. The gap may occur along a number of different locations in the fiber optical cables. Moreover, the first surface may be generally aligned with the corresponding second surface of the fiber optical cables so that at least a portion of the light emitted from the first surface of the fiber optical cables may pass through the gap and to the second surface, whereupon the light may continue to be transmitted to the distal end of the fiber optical cables. The first and second surfaces may be retained in alignment through the use of a connector. According to such embodiments, the gap may act as a resistor that assists in reducing or eliminating the amount of heat at the distal end of the fiber optical cables.

According to certain embodiments, the bundle of fiber optical cables may pass through a flexible conduit or tube, or sheath, before reaching the light housing. According to certain embodiments, the sheath may be operably connected to the light housing, such as, for example, through a mechanical connection, weld, or adhesive. The sheath may provide protection to the fiber optical cables. Further, the sheath may be turned, twisted, flexed, or otherwise manipulated, and retain a desired configuration so that light passing through the distal end of the optical cables may be directed to the desired location.

The surgical light apparatus may also be removably positioned on the surgical instrument by an attachment mechanism. The attachment mechanism may also include a passage that is sized to receive the insertion of at least a portion of the surgical instrument. According to such an embodiment, at least a portion of the attachment mechanism may be configured to exert a force against at least a portion of the surgical instrument located in the passage so as to enable the light housing to stay at a desired position on the surgical instrument.

According to some embodiments, the attachment mechanism may be integrally formed as part of the light housing. According to other embodiments, the attachment mechanism may be removably attached to the surgical light apparatus. According to such embodiments, the attachment mechanism may include a chamber that is sized to removably receive or mate with at least a portion the surgical light apparatus, such as being configured to be attached to the sheath. According to other embodiments, the attachment mechanism may be operably connected to the surgical light apparatus, such as through the use of a mechanical connection, including, for example, through the use of a snap, clasp, clip, retention arm, pin, or threaded connection, among others. Such embodiments may allow different types and/or sizes of attachment mechanisms to be used with the surgical light instrument. For instance, a retractor blade may require a different attachment mechanism than a suction tube assembly. By being able change the attachment mechanism, or change at least a portion of the attachment mechanism, the surgical light apparatus can be used with a variety of different surgical instruments.

### BRIEF DESCRIPTION OF SEVERAL VIEWS OF THE DRAWINGS

FIG. 1 illustrates a perspective view of a surgical light apparatus according to an embodiment of the present invention.

FIG. 2 illustrates a top cross-sectional view of a surgical light apparatus according to an embodiment of the present invention.

FIG. 3 illustrates a front view of a surgical light housing according to an embodiment of the present invention.

FIG. 4 illustrates a rear view of a surgical light housing according to an embodiment of the present invention.

FIG. 5 illustrates a front view of a cover of a surgical light housing according to an embodiment of the present invention.

FIG. 6 illustrates a cross-sectional view of a portion of a bundle of fiber optical cables having a gap between first surfaces and second surfaces of the cables according to an embodiment of the present invention.

FIG. 7 illustrates a top view of the main body of a surgical light housing according to an embodiment of the present invention.

FIG. 8 illustrates a side view of a surgical light apparatus according to an embodiment of the present invention.

FIG. 9 illustrates a front view of a light housing according to an embodiment of the present invention.

FIG. 10 illustrates a front view of an attachment mechanism according to an embodiment of the present invention.

FIG. 11 illustrates a side view of a portion of a surgical light apparatus having a surgical light housing attached to a retractor blade according to an embodiment of the present invention.

FIG. 12 illustrates a bottom view of a portion of a surgical light apparatus having a surgical light housing attached to a retractor blade according to an embodiment of the present invention.

FIG. 13 illustrates a top view of a portion of a surgical light apparatus having a surgical light housing attached to a retractor blade according to an embodiment of the present invention.

FIGS. 14a and 14b illustrate a side view and a front view, respectively, of a surgical light apparatus attached to a suction tube assembly, according to an embodiment of the present invention.

The foregoing summary, as well as the following detailed description of certain embodiments of the present invention, will be better understood when read in conjunction with the appended drawings. For the purpose of illustrating the invention, there is shown in the drawings, certain embodiments. It should be understood, however, that the present invention is not limited to the arrangements and instrumentalities shown in the attached drawings.

### DETAILED DESCRIPTION OF THE INVENTION

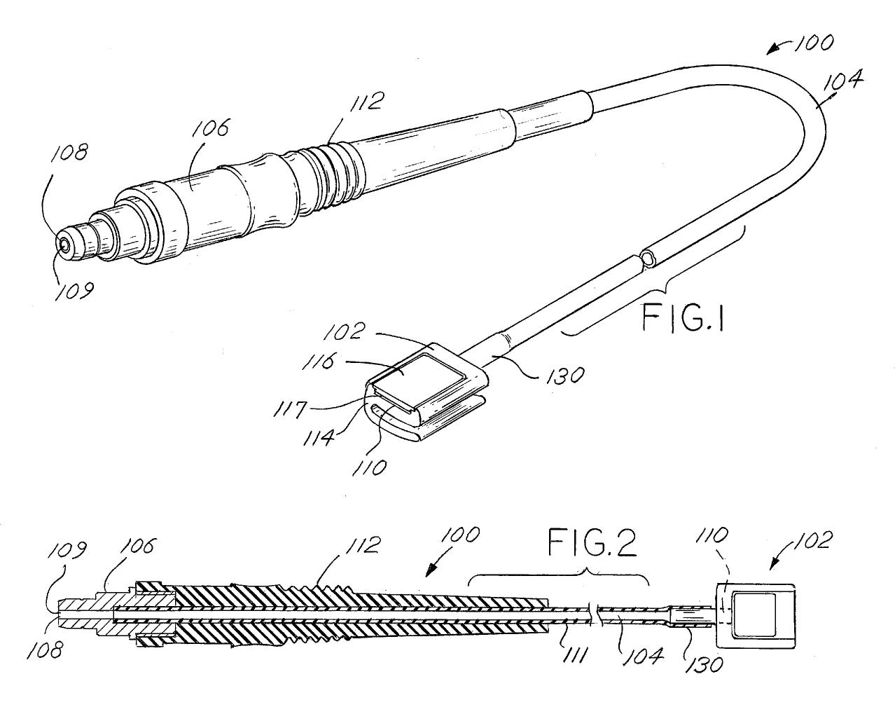

FIG. 1 illustrates a perspective view of a surgical light apparatus 100 according to an embodiment of the present invention. The surgical light apparatus 100 may include a surgical light housing 102, a bundle of fiber optical cables 104, and a cable connector 106. The bundle of fiber optical cables 104 may include a proximate end 108 and a distal end 110. According to certain embodiments, the bundle of fiber optical cables 104 may have a diameter of approximately 1 to 5 millimeters. Additionally, each fiber optical cable may include a core through which light may travel, an outer cladding, and a buffer coating. The core may be constructed for a variety of materials, including, for example, glass, plastic, or combination thereof, among others, and may be multi-mode or single mode fibers.

FIG. 2 illustrates a top cross-sectional view of a surgical light apparatus 100 according to an embodiment of the present invention. The connector 106 may be a standard fiber optical connector, such as, for example, a standard ACMI adapter. At least a portion of the proximate end 108 of the bundle of fiber optical cables 104 may be operably connected to the cable connector 106, for example through the use of bonding, crimping, mechanical connection, or adhesive, such as a surgical grade epoxy, among others. The cable connector 106 may also include, be adjacent to, or be operably connected to, a strain relief 112, such as by an adhesive or mechanical connection. The strain relief 112 may provide additional strength to the connection between the bundle of fiber optical cables 104 and the connector 106.

According to the embodiment shown in FIG. 2, the bundle of fiber optical cables 104 is a bundle of fiber optical cable includes an outer jacket 111. Moreover, the bundle of fiber optical cables 104 may be protected and/or joined together by the outer jacket 111. According to certain embodiments, the outer jacket 111 may be constructed from silicone. The proximate end 108 of the bundle of fiber optical cables 104 may extend beyond the terminated portion of the jacket 111 so that the proximate end 108 of the fiber optical cables 104 may be exposed through an orifice 109 at the end of the connector 106, as shown in FIG. 1. Moreover, the cores of the fiber optical cables 104 may be aligned at the end of the connector 106 so that at least a portion of the bundle of fiber optical cables 104 receive light transmitted from a light source and/or from another cable that is operably connected to the cable connector 106. Further, at least a portion of the proximate end 108 of the fiber optical cables 104 may be polished.

The cable connector 106 may be operably connected to a standard lamp housing (not shown). The lamp housing may include a connector that may mate with, or be coupled to, the cable connector 106, or other cable that is operably connected to the cable connector 106. For example, the cable connector 106 and the connector of the lamp housing, or other cable that is operably connected to the lamp housing, may be configured for a mechanical male-female connection. Alternatively, the connector of the lamp housing may be a coupling apparatus that joins with the cable connector 106.

The lamp housing may include a light source, such as a light bulb, among others. For example, according to certain embodiments, the light source may be a 300 watt xenon light bulb. The light source may be powered by a power source, for example by electrical utility power or one or more than one battery, among others. Further, the lamp housing may include a light illumination control, which may control the power supplied to the light bulb, and thus control the intensity of the illumination emitted by the light source.

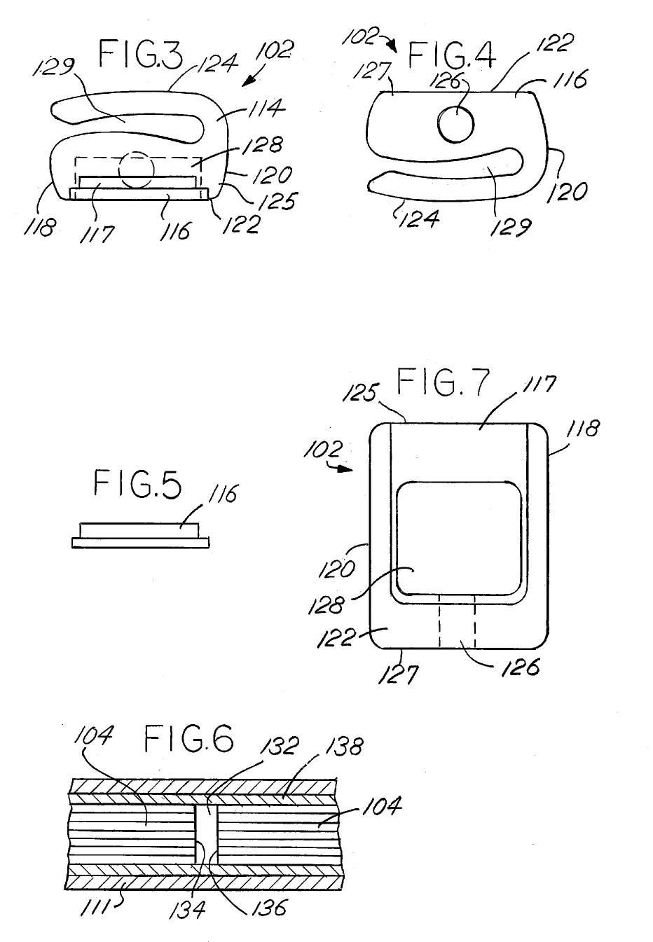

FIGS. 3 and 4 illustrate a front view and a rear view, respectively, of a surgical light housing 102 according to an embodiment of the present invention. The surgical light housing 102 may have a unitary construction or may include a main body 114 and a cover 116. Further, the surgical light housing 102 may be constructed from a variety of materials, including a surgical grade material, such as surgical grade plastic or nylon, or a radio opaque material, among others.

The main body 114 of the surgical light housing 102 illustrated in FIGS. 3 and 4 may include a first sidewall 118, a second sidewall 120, a top portion 122, a bottom portion 124, a front wall 125, and a rear wall 127. At least a portion of the top portion 122 may be configured to receive the placement of at least a portion of the cover 116 against the main body 114. The surgical light apparatus 100 may also include an attachment mechanism that may be integrated into, or may be operably attached to, the surgical light housing 102, for example, by a mechanical connection, such as a clip, clasp, retention arms, pin, interference fit, or threadable mount, among others. The attachment mechanism may allow the surgical light housing 102 to be attached to at least a portion of a surgical instrument, such as a retractor blade, retractor frame, or suction tube, among others. For example, according to certain embodiments, the attachment mechanism may be a passage 129 formed in or by the main body 114, that is configured to attach, such as clip, the surgical light housing 102 to the surgical instrument.

The main body 114 may also include an aperture 126 that is configured to receive the insertion of at least a portion of the bundle of fiber optical cables 104. Further, the bundle of fiber optical cables 104 may be operably connected to the surgical light housing 102, such as though the use of an adhesive, for example a surgical grade epoxy, and/or a mechanical connection. Further, the bundle of fiber optical cables 104 may pass through, or be attached to, a tube 130, as shown in FIGS. 1 and 2, that is operably connected to the aperture 126 of the surgical light housing 102. According to certain embodiments, the tube 130 may be a second strain relief that may assist in retaining the connection between the bundle of fiber optical cables 104 and the surgical light housing 102.

FIG. 5 illustrates a front view of a cover 116 of a surgical light housing 102 according to an embodiment of the present invention. The cover 116 may be constructed from the same or different material as the main body 114. Further, the cover 116 and/or main body 114 may be configured to allow for the secure placement of the fiber optical cables 104 in the surgical light housing 102. The cover 116 may be operably connected to the main body 114 through a mechanical connection, such as by the use of screws, pins, tabs, or interference fit, among others, or may be joined by an adhesive, such as by a surgical grade epoxy.

The cover 116 and/or main body 114 may be configured so that, when operably joined together, an outlet having a slot 117 configuration is provided along at least a portion of the front wall 125, as illustrated in FIGS. 1 and 3. The slot 117 may be configured to contain and/or expose at least a portion of the distal ends 110 of the bundle of fiber optical cables 104. Moreover, the distal ends 110 of the bundle of fiber optical cables 104 may be oriented in or about the slot 117 so that at least a portion of the light transmitted along the bundle of fiber optical cables 104 is emitted away from the surgical light housing 102. The position and/or orientation of the distal end 110 of the bundle of fiber optical cables 104 may be secured in the surgical light housing 102 through the use of mechanical connection and/or adhesives, such as a surgical grade epoxy. Further, at least a portion of the distal ends 110 of the bundle of fiber optical cables 104 exposed at the slot 117 may be polished.

As shown by the embodiment in FIGS. 1 and 3, the slot 117 may have a generally rectangular or linear configuration, for example, being approximately 0.250 inches long by approximately 0.0060 inches wide. According to such an embodiment, at least a portion of the distal ends 110 of the fiber optical cables 104 may be spread about at least a portion of the slot 117. However, a variety of different configurations may be used for the slot, including, but not limited to, circular, square, trapezoidal, triangular, non-linear, and/or combination thereof, among others. Further, rather than be one continuous opening, the slot 117 may be comprised of a plurality of openings that may or may not be joined together.

According to certain embodiments, the slot 117 may be configured to create dead space around, or between the fiber optical cables 104, and more particularly between the cables in the bundle of fiber optical cables 104. For example, according to embodiments in which the bundle of fiber optical cables 104 has a diameter of about 2 millimeters, the optical fiber cables 104 may be spread about at least a portion of a slot 117 that is approximately 0.250 inches long by approximately 0.012 inches wide. The relatively large size of the slot 117 for the bundle of fiber optical cables 104 may result in larger or more spaces being created along the slot 117 that are not occupied by fiber optical cable 104. The spreading of the bundle of fiber optical cables 104 about the slot 117 may assist in dispersing the heat associated with light being emitted from the distal ends 110 of the bundle of fiber optical cables 104. The dispersal of such heat may allow for a lower operating temperature about the distal end 110 of the fiber optical cables 104 and/or the front wall 125 of the surgical light housing 102. Moreover, the reduced temperature may permit the surgical light housing 102 to be placed in close proximity to the surgical site with a reduced risk of the heat emitted from the distal end 110 burning the patient.

According to certain embodiments, at least a portion of the spaces between the dispersed fiber optical cables 104 may be filled or at least partially occupied by filler material, for example glass, plastic, or diamond based fillers, among others. Along with occupying these spaces, the filler may also assist with the dispersal of the bundle of optical fiber cables 104, and thereby assist in dispersing the heat associated with transmitting light from the distal end of the fiber optical cable.

FIG. 6 illustrates a cross-sectional view of a portion of a bundle of fiber optical cables 104 having a gap 132 between first surfaces 134 and second surfaces 136 of the cables 104 according to an embodiment of the present invention. The inclusion of a gap 132, or air space, at a location between the proximate end 108 and the distal end 110 of the fiber optical cables 104 may assist in releasing at least some heat transmitted or generated by the fiber optical cables 104, and thereby reduce the temperature at the distal end of the fiber optical cables 104. Moreover, the gap 132 may act as a resistor that assists in reducing or eliminating heat at the distal end of the fiber optical cables 104. According to certain embodiments, the gap 132 may be the form of an air space between adjacent first 134 and second surfaces 134, 136 of the fiber optical cables 104.

The gap 132 may be sized so that heat may be released between the first and second surfaces 134, 136 may be dispersed, while also allowing for light to be transmitted from the first surfaces 134, through the gap 132, and to the second surfaces 136. Moreover, the first surfaces 134 may be generally aligned with the corresponding second surfaces 136 of the fiber optical cables 104 so that at least a portion of the light emitted from the fiber optical cables 104 at the first surfaces 134 may pass through the gap 132 and to the second surfaces 136, whereupon the light may continue traveling through the fiber optical cables 104 to the distal end 110 of the cables 104. The first and second surfaces 134, 136 may be retained in alignment through the use of a connector 138, such as a tube, cable adapter, or cable connector that is used to connect or couple fiber optical cables together, among others. According to certain embodiments, the connector 138 may be positioned within the jacket 111 of the fiber optical cables 104, outside of the jacket 111, or may abut against at least a portion of fiber optical cables 104 that do not have a jacket 111 or have had the jacket 111 removed.

FIG. 7 illustrates a top view of the main body 114 of a surgical light housing 102 according to an embodiment of the present invention. According to certain embodiments, the main body 114 may include a cavity 128, as shown in FIGS. 3 and 7, which may be configured to receive the placement of at least a portion of the fiber optical cables 104. Moreover, the cavity 128 may be sized so as to allow for the routing of the distal end 110 of the fiber optical cables 104 through the slot 117.

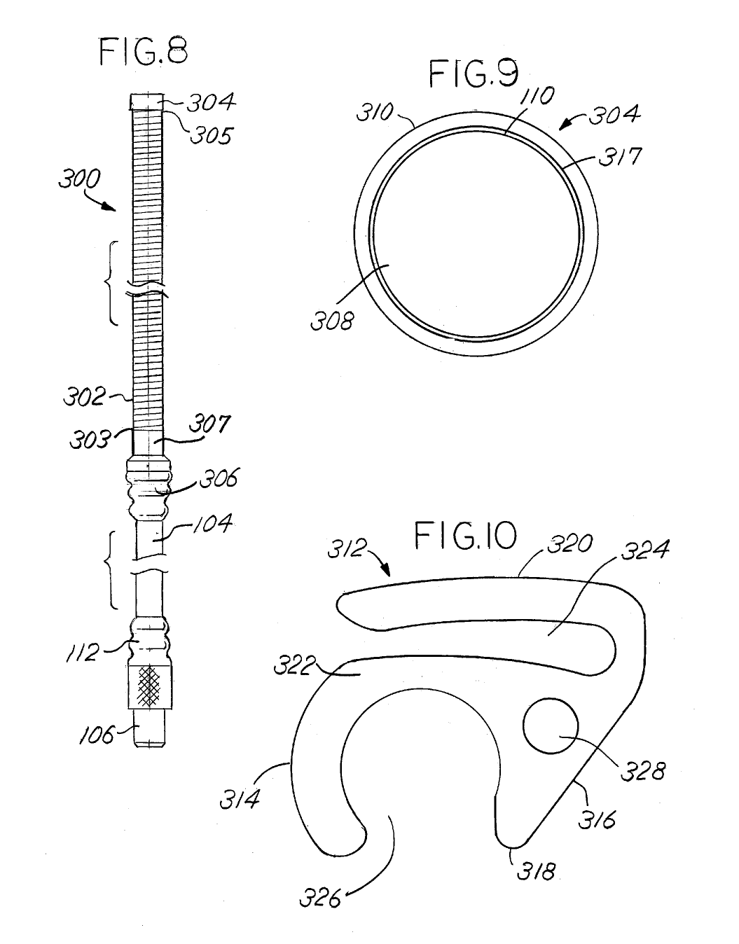

FIG. 8 illustrates a side view of a surgical light apparatus 300 according to an embodiment of the present invention. The surgical light apparatus 300 may include a cable connector 106, a first strain relief 112, a bundle of fiber optical cables 104, a sheath 302, and a light housing 304. Again, as discussed above, the proximate end 108 of the bundle of fiber optical cables 104 may be operably connected to the cable connector 106 and a strain relief 112.

According to certain embodiments, the sheath 302 may be a flexible conduit or tubing, such as, for example, a flexible metal conduit that includes a helically wound metal strip having square locked or interlocked construction, among others. According to some embodiments, the sheath 302 is constructed from stainless steel. The sheath includes a proximate end 303 and a distal end 305. At least a portion of bundle of the fiber optical cables 104 passes through the sheath 302. However, the bundle of fiber optical cables 302 may or may not be attached to the sheath 302. For example, in some embodiments in which the bundle of fiber optical cables 104 are attached to the sheath 302, an epoxy, such as for example a surgical grade epoxy, may be used to operably attach the jacket 111 to the proximate 303 and/or distal end 305 of the sheath 302.

According to certain embodiments, the proximate end 303 of the sheath 302 may be operably connected to a collar 307, such as, for example, by being soldered, welded, a mechanical connection, or adhered. Alternatively, the sheath 302 may be formed or constructed to include the collar 307. The collar 307 may also be operably attached to a second strain relief 306, which may be operably connected to the bundle of fiber optical cables 104, such as, for example, through an epoxy, among others. According to some embodiments, the collar 307 may be soldered to the second strain relief 306.

FIG. 9 illustrates a front view of a light housing 304 according to an embodiment of the present invention. The light housing 304 may be operably connected to the distal end 305 of the sheath 302, such as, for example, by a mechanical connection, weld, epoxy, or solder. The surgical light housing 304 may be constructed from a variety of materials, including, for example, a surgical grade material, such as surgical grade plastic or nylon, or stainless steel, or a combination thereof, among others. Additionally, the light housing 304 may or may not have a unitary body construction or may be include a main body 308 and a cover 310. For example, the embodiment shown in FIG. 13 may include a generally circular shaped solid main body 308, around which the distal ends 110 of the fiber optical cables 104 may be dispersed, and a cover 308 that is configured to fit over at least a portion of the main body 308 and fiber optical cables 104. The cover 310 may provide for, in combination with the main body 308 allow for, the formation of the outlet 317, through which light may be transmitted from the distal ends 110 of the fiber optical cables 104. And, as previously mentioned, the distal ends 110 of the fiber optical cables may be dispersed about, and filler material may be included, in the area of the outlet 317, so as assist in dispersing heat generated by the light emitted through and/or from the fiber optical cables 104. Additionally, the main body 308 or cover 310 may include, or be operably connect to (such as through the use of an epoxy or mechanical connection, among others) a lens. Additionally, the distal ends 110 of the fiber optical cable 104 may be polished.

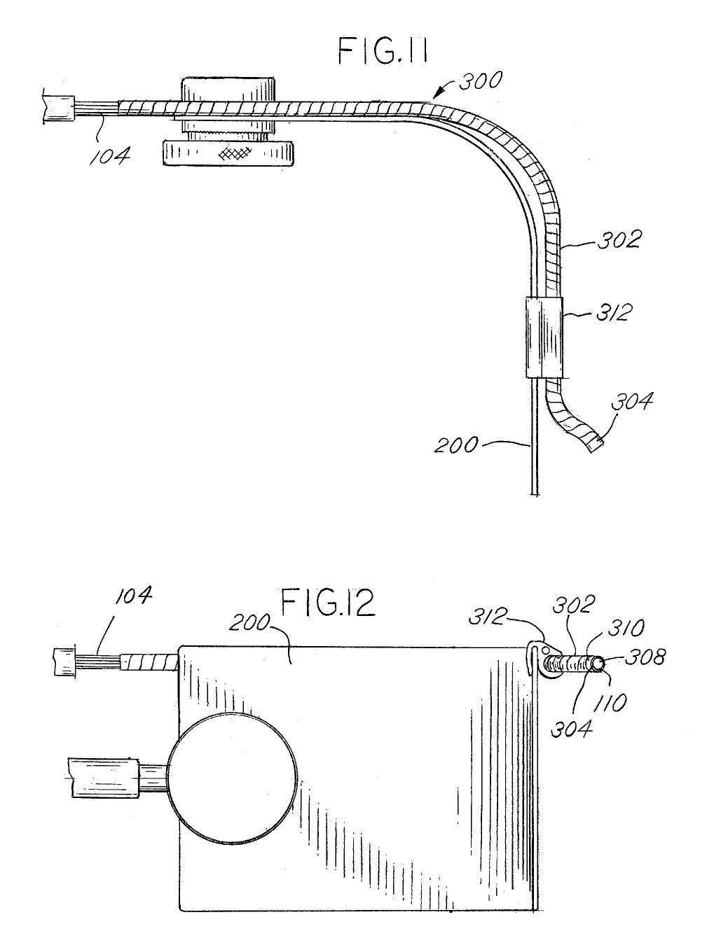

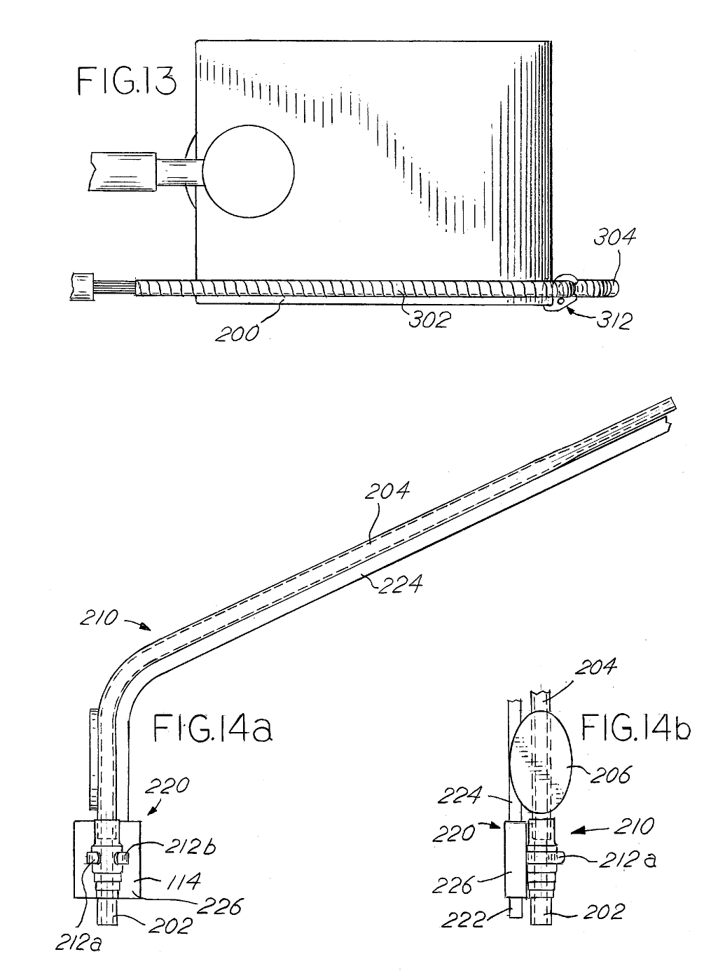

FIG. 10 illustrates a front view of an attachment mechanism 312 according to an embodiment of the present invention. The attachment mechanism 312 includes a first sidewall 314, a second sidewall 316, a top portion 318, a bottom portion 320, a front wall 322, and a rear wall (not shown). The attachment mechanism 312 also includes a passage 324 that is configured allow the attachment mechanism to be connected to a surgical apparatus. For example, in the embodiment illustrated in FIG. 14, the passage 324 is configured to receive the placement of a retractor blade (not shown). Further, the configuration of the passage 324 and/or material selected for the attachment mechanism 312 may allow at least a portion of the attachment mechanism 312 to exert a force against at least a portion of the surgical instrument placed in the passage 324 so that the attachment mechanism 312 may be securely placed at a desired position on the surgical instrument.

The attachment mechanism 312 also includes a chamber 326 that is configured to receive the removable placement of at least a portion of the light apparatus 300. For example, the chamber 326 may be configured to receive the placement of the sheath 302 of the surgical light apparatus 300. According to such an embodiment, the chamber 326 may be sized so that, when the light apparatus 300 is placed in the chamber 326, at least a portion of the attachment mechanism 312 exerts a force on the adjacent portion of the light apparatus 300, and thereby may securely attach the attachment mechanism 312 to the light apparatus 300. However, as previously discussed, the attachment mechanism 312 may be connected to the surgical light apparatus 300 by a variety of different mechanical connections.

The attachment mechanism 312 may also be constructed to be detectable by x-ray in the event the clip is lost of forgotten in a patient. For example, the attachment mechanism may be constructed from a radio opaque material that may be detected or shown on an x-ray image. Alternatively, the attachment mechanism may be include an x-ray detectable attachment, such as a stainless steel plate or pin that may fit against, or into the attachment mechanism 312. For example, a stainless steel pin may be placed in a hole 328 that may run through a part of, or all of, the attachment mechanism 312.

FIGS. 11, 12, and 13 illustrate side, bottom, and top views, respectively, of a portion of a surgical light apparatus 300 attached to an attachment mechanism 312 that is attached to a retractor blade 200 according to an embodiment of the present invention. The retractor blade 200 may be designed to hold back a patient's anatomy in the immediate area of the operative site, thereby enabling a surgeon to have both an optimal view of the operative site and a sufficiently opened area within which to work. As previously mentioned, according to certain embodiments of the present invention, the attachment mechanism 312 may be attached to the retractor blade 200 by placing at least a portion of the retractor blade 200 in the passage 324 of the attachment mechanism 312.

The attachment mechanism 312 may be secured at a number of locations along the light apparatus 300. For example, as shown in FIGS. 11, 12, and 13, at least a portion of the sheath 302 may be positioned, and retained, in the chamber 326 of the light apparatus. Additionally, the attachment mechanism 312 may be attached to the sheath 302 at a sufficient distance away form the light housing 304 so as to enable the portion of the sheath 302 between the attachment mechanism 312 and light housing 304 to be manipulated to direct the light emitted from the distal ends 110 of the fiber optical cables 104 in a plane different than that of the retractor blade 200. For example, the attachment mechanism 312 may be placed on the sheath 302 at least one inch away from the light housing 304 so that the portion of the sheath 304 between the attachment mechanism 312 and light housing 304 may be turned, twisted, or bent so that light being emitted from the distal end 110 of the fiber optical cables 110 is directed toward a desired location.

The attachment mechanism 312 may also be attached at a variety of locations along the retractor blade 200. For example, during surgery, moving the attachment mechanism 312 up on the vertical portion of the retractor blade 200 may expand the size of the area of the surgical site that is illuminated by the light emitted from the distal end 110 of the fiber optical cables 104. Alternatively, moving the attachment mechanism 312 down the vertical portion of the retractor blade 200 may concentrate the light emitted from the distal end 110 of the fiber optical cables 104 to a smaller area of the surgical site, which may also increase the brightness of the smaller illuminated area.

FIGS. 14a and 14b illustrate a side view and a front view, respectively, of a suction tube light apparatus 220 attached to a suction tube assembly 210, according to an embodiment of the present invention. The suction tube light apparatus 220 may include a light housing 222, fiber optical cable 224, and a cable connector 106. The suction tube assembly 210 may include a suction head 202, a suction tube 204, and a grasp 206. The light housing 222 may include, or be operably connected to, an attachment mechanism 226 that may be attached to the suction tube assembly 210 at a variety of locations. For example, as shown in. FIGS. 14a and 14b, the attachment mechanism 226 of the suction tube light apparatus 220 may include retention arms 212a, 212b that may be placed over at least a portion of the suction head 202. Moreover, the retention arms 212a, 212b may be configured so that, when positioned about at least a portion of the suction tube assembly 210, the retention arms 212a, 212b exert, or are caused to exert, a generally inwardly force so as to securely retain the suction tube light apparatus 220 at a desired position. Alternatively, the attachment mechanism 226 may be configured to allow the surgical light apparatus 220 to be attached to the suction tube 204, among other locations by a variety of different mechanical connections, among others, including for example, a snap, clasp, clip, pin, or threaded connections, among others

While the embodiments of the surgical light apparatuses of the present invention have been discussed above with relation to embodiments used with retractor blades 200 and suction tube assemblies 210, the surgical light apparatus of the present invention may be used with a variety of different surgical instruments, including clamps, drills, and powered saws, among others.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A surgical light apparatus comprising:
a bundle of fiber optical cable, the bundle of fiber optical cable having a distal end and a proximate end, the proximate end configured to receive light emitted from a light source, at least a portion of the light received at the proximal end being transmitted through the bundle of fiber optical cable, the distal end configured to emit at least a portion of the transmitted light;
a cable connector, the cable connector operably connected to the proximate end of the bundle of fiber optical cable, the cable connector configured to receive light from a light source;
a light housing, the light housing having an outlet, the outlet being configured to receive the placement and disbursement of at least a portion of the distal end of the bundle of fiber optical cable about the outlet; and
an attachment mechanism, the attachment mechanism configured to operably connect the surgical light apparatus to at least a portion of a surgical instrument.

2. The surgical light apparatus of claim 1 further including a sheath, a sheath, the sheath covering at least a portion of the bundle of fiber optical cable, the sheath configured to be able to be manipulated to retain a configuration that directs light emitted from the distal end of the fiber optical cable to a desired location.

3. The surgical light apparatus of claim 2 wherein the attachment mechanism includes a passage, the passage configured to clip the light housing onto at least a portion of a retractor blade.

4. The surgical light apparatus of claim 2 wherein the attachment mechanism includes a passage the passage configured to clip the light housing onto at least a portion of a retractor frame.

5. The surgical instrument of claim 3 wherein the attachment mechanism includes a chamber, the chamber configured to mate with at least a portion of the sheath to securely attach the attachment mechanism to the surgical instrument.

6. The surgical light apparatus of claim 1 wherein at least a portion of the outlet is occupied by a filler material.

7. The surgical light apparatus of claim 1 wherein the bundle of fiber optical cable has a gap between a first surface and an second surface, the first surface and second surface being oriented to allow transmitted light to pass from the first surface, through the gap and to the second surface before the transmitted light travels to the distal end of the bundle of fiber optical cable.

8. The surgical light apparatus of claim 1 wherein the outlet has a slot configuration.

9. A surgical light apparatus comprising:
a fiber optical cable, the fiber optical cable having a distal end and a proximate end, the proximate end configured to receive light emitted from a light source, at least a portion of the light received at the proximal end being transmitted through the fiber optical cable, the distal end configured to emit at least a portion of the transmitted light;
a cable connector, the cable connector operably connected to the proximate end of the fiber optical cable;
a surgical light housing, the surgical light housing having an outlet, the outlet being configured to receive the placement and disbursement of at least a portion of the distal end of the fiber optical cable;
a sheath, the sheath covering at least a portion of the bundle of fiber optical cable, the sheath configured to be able to be manipulated to retain a configuration that directs light emitted from the distal end of the fiber optical cable to a desired location; and
an attachment mechanism, the attachment mechanism including a passage, the passage being configured to clip the surgical light apparatus to a surgical instrument.

10. The surgical light apparatus of claim 9 wherein the light source is a xenon light bulb.

11. The surgical light apparatus of claim 12 wherein at least a portion of the outlet is occupied by a filler material.

12. The surgical light apparatus of claim 2 wherein the attachment mechanism includes a passage, the passage configured to clip the light housing onto at least a portion of a retractor blade.

13. The surgical light apparatus of claim 2 wherein the attachment mechanism includes a passage, the passage configured to clip the light housing onto at least a portion of a retractor frame.

14. The surgical light apparatus of claim 13 wherein the fiber optical cable has a gap between a first surface and an adjacent second surface.

15. The surgical light apparatus of claim 1 wherein the outlet has a slot configuration.

16. A surgical light apparatus comprising:
a fiber optical cable comprised of a bundle of fiber optical cable, the fiber optical cable having a distal end and a proximate end, the proximate end configured to receive light emitted from an xenon light source, at least a portion of the light received at the proximal end being transmitted through the fiber optical cable, the distal end configured to emit at least a portion of the transmitted light;
a cable connector, the cable connector operably connected to the proximate end of the fiber optical cable; and
a surgical light housing, the surgical light housing having a main body, an outlet, the outlet being configured to receive the placement and disbursement of at least a portion of the distal end of the fiber optical cable;
a sheath, the sheath covering at least a portion of the bundle of fiber optical cable, the sheath configured to be able to be manipulated to retain a configuration that directs light emitted from the distal end of the fiber optical cable to a desired location;
and an attachment mechanism, the attachment mechanism including a passage and a chamber, the passage being configured to clip the surgical light apparatus to at least a portion of a surgical instrument, the chamber configured to receive the retainable insertion of at least a portion of the sheath.

17. The surgical light apparatus of claim 16 wherein the surgical light housing includes a main body and a cover.

18. The surgical light apparatus of claim 16 wherein attachment mechanism is constructed of a radio opaque material.

19. The surgical light apparatus of claim 16 wherein the attachment mechanism includes an x-ray detectable attachment.

20. The surgical light apparatus of claim 16 wherein the fiber optical cable has a gap between a first surface and an second surface, the first surface and second surface being oriented to allow transmitted light to pass from the first surface, through the gap and to the second surface before the transmitted light travels to the distal end of the fiber optical cable.
